(19) United States
(12) Patent Application Publication
(10) Pub. No.: US 2015/0282935 A1
(43) Pub. Date: Oct. 8, 2015

(54) INTRAMEDULLARY DEVICE FOR TREATING PERIPROSTHETIC FRACTURES

(71) Applicant: Biomet Trauma, LLC, Warsaw, IN (US)

(72) Inventors: Djoldes Kuldjanov, Town and Country, MO (US); Paul D'Antonio, Winona Lake, IN (US); Joseph Michael O'Reilly, Granger, IN (US); Adam Finley, Leesburg, IN (US)

(73) Assignee: Biomet Trauma, LLC, Warsaw, IN (US)

(21) Appl. No.: 14/574,477

(22) Filed: Dec. 18, 2014

Related U.S. Application Data
(60) Provisional application No. 61/975,154, filed on Apr. 4, 2014.

(51) Int. Cl. A61F 2/36; A61F 2/38

(52) U.S. Cl. CPC A61F 2/3662; A61F 2/3609; A61F 2/3859; A61F 2002/3692; A61F 2002/3652; A61F 2210/0014; A61F 2002/4631

(57) ABSTRACT

An intramedullary device comprises a tubular body extending along a center longitudinal axis. The tubular body includes a first end and a second end. The first end is configured to engage a stem of an implant.

### CROSS-REFERENCE TO RELATED APPLICATIONS

This application claims the benefit of U.S. Provisional Application No. 61/975,154, filed on Apr. 4, 2014. The entire disclosure of the above application is incorporated herein by reference.

### FIELD

The present disclosure generally relates to an intramedullary device for treating periprosthetic fractures.

### BACKGROUND

This section provides background information related to the present disclosure and is not necessarily prior art.

Fractures termed periprosthetic fractures may occur in patients proximate to joint replacement implants. Such fractures may occur intraoperatively or postoperatively. Periprosthetic fractures may be conventionally treated using nails, screws, plates, and cables. While known treatments have proven to be generally acceptable, a continued need for improvement in the relevant art remains.

### SUMMARY

This section provides a general summary of the disclosure, and is not intended to be a comprehensive disclosure of its full scope or all of its features.

According to one particular aspect, the present disclosure provides an intramedullary device including a tubular body extending along a center longitudinal axis. The tubular body includes a first end and a second end. The first end is configured to engage a stem of an implant.

According to another particular aspect, the present disclosure provides an implantable construct including a knee implant, a hip implant having an intramedullary hip stem and an intramedullary tube. The intramedullary tube extends along a longitudinal axis from a proximal end to a distal end. The proximal end of the intramedullary tube includes a cavity configured to receive and engage a distal end of the intramedullary hip stem therein. The distal end of the intramedullary tube is configured to engage the knee implant.

According to yet another particular aspect, the present disclosure provides a method for fixating a bone. The method includes providing a tubular member; inserting the tubular member into an intramedullary canal of a patient; and engaging a first end of the tubular member with a stem of an implant while the stem is in the intramedullary canal of the patient.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a perspective view of an exemplary construct including an intramedullary device in accordance with the teachings of the present disclosure, the exemplary construct is further shown to include a hip stem and a knee femoral component;

FIG. 2 is an exploded perspective view of the construct of FIG. 1;

FIG. 3 is a cross-sectional view of the intramedullary device of the present teachings taken along the line 3-3 of FIG. 2;

FIG. 4 is an exploded, partial cross-sectional view of another exemplary construct including an intramedullary device in accordance with the teachings of the present disclosure, the exemplary construct is further shown to include a hip stem;

FIG. 5 is a partial cross-sectional view of the exemplary construct of FIG. 4, the exemplary construct is further shown operatively associated with a cement gun;

FIG. 6A is an exploded, partial cross-sectional view of another exemplary construct including an intramedullary device in accordance with the teachings of the present disclosure, the exemplary construct is further shown to include a distal tip of a hip stem;

FIG. 6B is a partial cross-sectional view of the exemplary construct of FIG. 6A, the exemplary construct is shown to include the distal tip of the hip stem received in the intramedullary device in accordance with the teachings of the present disclosure;

FIG. 6C is another partial cross-sectional view of the exemplary construct of FIG. 6A, the exemplary construct is shown to include the distal tip of the hip stem received in the intramedullary device in accordance with the teachings of the present disclosure;

FIG. 6D is a cross-sectional view of the construct of FIG. 6C taken along the line 6D-6D of FIG. 6C;

FIG. 7A is an exploded, partial cross-sectional view of another exemplary construct including an intramedullary device in accordance with the teachings of the present disclosure, the exemplary construct is further shown to include a distal tip of a hip stem;

FIG. 7B is a partial cross-sectional view of the exemplary construct of FIG. 7A, the exemplary construct is shown to include the distal tip of the hip stem received in the intramedullary device in accordance with the teachings of the present disclosure; and

FIG. 7C is another partial cross-sectional view of the exemplary construct of FIG. 7A, the exemplary construct is shown to include the distal tip of the hip stem received in the intramedullary device in accordance with the teachings of the present disclosure.

Corresponding reference numerals indicate corresponding parts throughout the several views of the drawings.

### DETAILED DESCRIPTION

The following description is merely exemplary in nature and is in no way intended to limit the invention, its application, or uses. For example, although the present description describes treatment of a periprosthetic fracture in a patient's femur, the present teachings can also be used to treat fractures in other bones. It will be understood that general surgical procedures are outlined only as needed to illustrate the devices and methods provided by the present teachings, while detailed descriptions of standard and known procedures and instruments are omitted for simplicity. Example embodiments will now be described more fully with reference to the accompanying drawings.

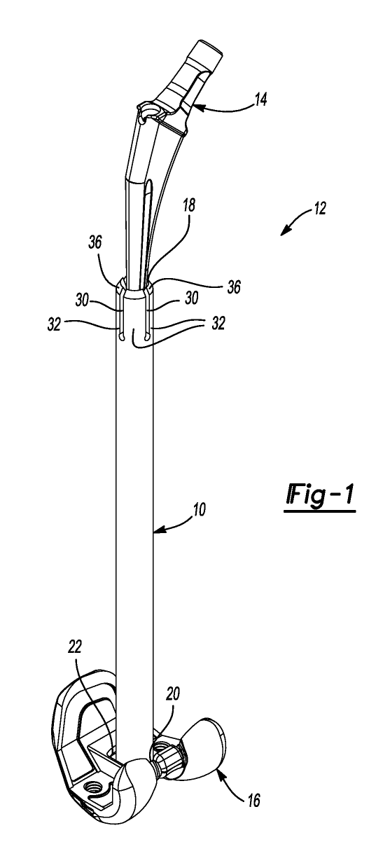

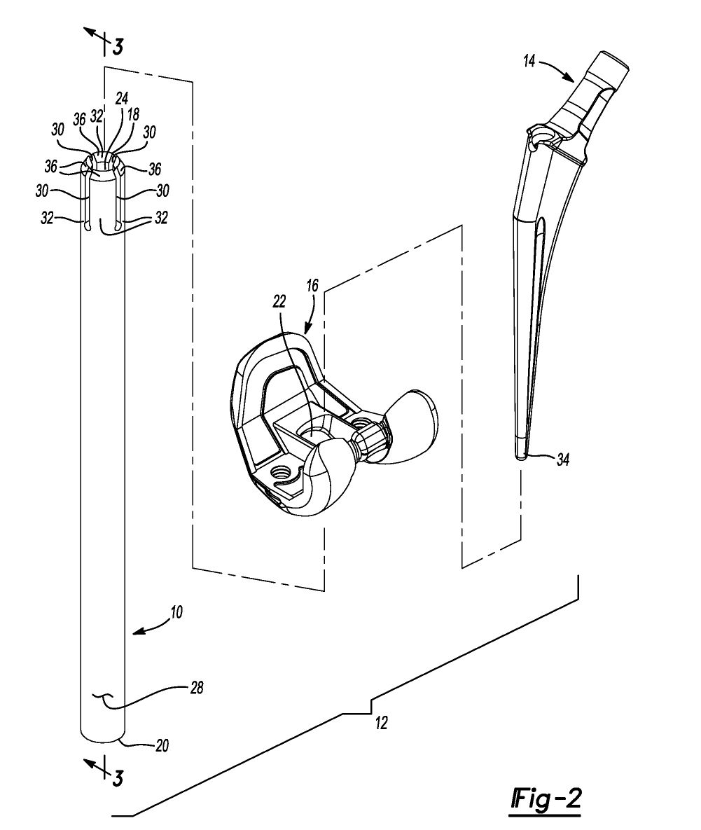

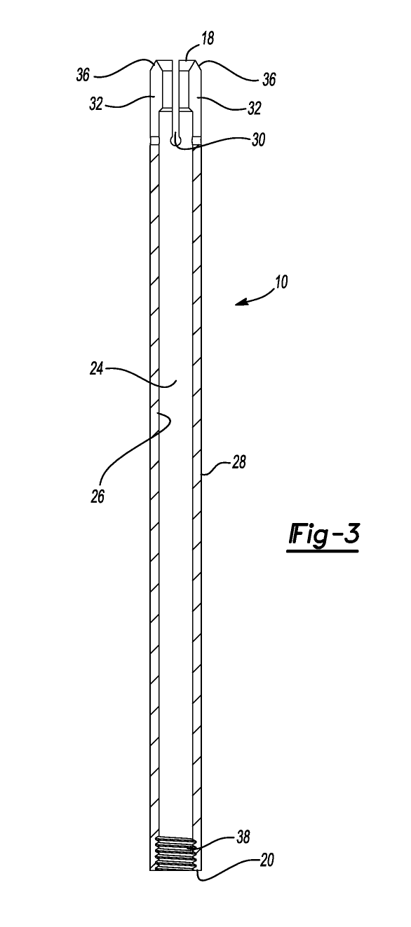

With initial reference to FIGS. 1-3, an intramedullary device for treating periprosthetic fractures constructed in accordance with the teachings of the present disclosure is illustrated and generally identified at reference numeral 10. In FIGS. 1 and 2, the intramedullary device 10 is shown incorporated into an exemplary construct 12. In this particular exemplary construct 12, the intramedullary device 10 is shown operatively associated with a hip stem 14 and a knee femoral component 16. Insofar as the present teachings are concerned, it will be understood that the hip stem 14 and the knee femoral component 16 are conventional in construction to any extent not otherwise described herein. It will be further understood that the present teachings may be used in connection with other implants and that the present teachings have application beyond the exemplary construct 12 illustrated in the drawings and described herein.

The intramedullary device 10 is an elongated member extending between a proximal end 18 and a distal end 20. According to one exemplary and non-limiting use, the intramedullary device 10 and the other intramedullary devices described below may be configured for insertion into the intramedullary canal of a patent's femur (not illustrated). According to other exemplary and non-limiting uses, the intramedullary device 10 and the other intramedullary devices described below may be configured for insertion into the intramedullary canal of other bones, including, but not limited to, the tibia and the humerus, for example.

With specific reference to FIGS. 1 and 2, the proximal end 18 of the intramedullary device 10 may be configured to engage the hip stem 14. The distal end 20 of the intramedullary device 10 may be configured to pass through an opening 22 in the knee femoral component 16. In this manner, the intramedullary device 10 may be implanted while the knee femoral component 16 is attached to the distal end of the femur. The proximal end 18 of the intramedullary device 10 may be correspondingly configured to engage other orthopedic implants, such as a tibial tray component or a shoulder stem. In these other applications, the distal end 20 of the intramedullary device 10 may be configured to pass through or attach to other orthopedic implants.

With continued reference to FIGS. 1-3, the intramedullary device 10 of the present disclosure may be fabricated from any material suitable for implantation and having requisite strength requirements including, but not limited to metal, ceramic, polymeric materials, and combinations thereof. A center bore 24 may extend longitudinally through the intramedullary device 10 between the proximal and distal ends 18, 20, thereby defining an inner channel or cavity 26 and an outer surface 28. The cavity 26 may include a cross section having a circular shape, or the cross section may include a different shape, such as an ovoid shape for example. Similarly, the outer surface 28 may include a cross section having a circular shape, or the cross section may include a different shape. Further, between the ends 18, 20, the intramedullary device 10 may include bends and/or other geometry that generally corresponds to the anatomical shape of the intramedullary canal. Accordingly, the overall geometry of the intramedullary device 10, including the cross section, length, and width may vary depending on its intended use.

At least one through bore (not illustrated) may extend through the intramedullary device 10 in a generally perpendicular or generally angular direction with respect to the longitudinally extending center bore 24. Once the intramedullary device 10 is inserted into the intramedullary canal of a patient's femur, the through bore(s) may provide for a bone fixation device(s), such as a plate, rod, screw, nail, etc., for example, to pass through the intramedullary device 10 and provide additional femoral fixation.

At the proximal end 18, the intramedullary device 10 may include a plurality of slots 30 cooperating to form a plurality of integral fingers 32. As perhaps best shown in the exploded view of FIG. 2, the intramedullary device 10 may include four slots 30 cooperating to form four fingers 32. It will be understood, however, that the intramedullary device 10 may include any number of slots 30 and fingers 32 within the scope of the present teachings. Alternatively, the fingers 32 may be rigidly attached to the proximal end 18 of the intramedullary device 10.

As will be described further below, as the intramedullary device 10 is inserted into the intramedullary canal of a patient's femur, the fingers 32 may radially deflect in a radial direction to engage and capture a distal end 34 of the hip stem 14 within the cavity 26. Each finger 32 may include a tapered end 36, which may assist in inserting the intramedullary device 10 into the intramedullary canal and/or assist in engaging and capturing the distal end 34 of the hip stem 14 within the cavity 26. The intramedullary device 10 may also include features within the cavity 26, such as a push rod or a balloon, for example (not illustrated), to assist in radially deflecting the fingers 32 open to capture the distal end 34 of the hip stem 14 within the cavity 26.

At the distal end 20, the intramedullary device 10 may include a threaded portion 38. A fastening means (not illustrated) may engage the threaded portion 38 and/or the intramedullary device 10 to attach the intramedullary device 10 to the knee femoral component 16. An insertion instrument (not illustrated) may be configured to engage the threaded portion 38 and/or the distal end 20 of the intramedullary device 10 to facilitate insertion of the intramedullary device 10 into the intramedullary canal of a patient's femur. The insertion instrument may also be configured to actuate the features within the cavity 26 (i.e., the push rod or the balloon describe above, for example) to radially deflect the fingers 32 open to capture the distal end 34 of the hip stem 14 within the cavity 26.

With continued reference to FIGS. 1-3, a method for treating a periprosthetic fracture in the femur of a patient having an existing hip stem 14 and knee femoral component 16 will be described. Using the insertion instrument for example, the proximal end 18 of the intramedullary device 10 may be inserted in a retrograde direction through the opening 22 in the knee femoral component 16 and into the intramedullary canal of the patient's femur. As the intramedullary device 10 is inserted into the intramedullary canal and the fingers 32 make contact with the distal end 20 of the hip stem 14, pressure applied by the hip stem 14 against the fingers 32 may cause the fingers 32 to outwardly deflect in a radial direction. Accordingly, as the intramedullary device 10 is further inserted into the intramedullary canal, the fingers 32 may engage and capture the distal end 34 of the hip stem 14 within the cavity 26. Alternatively, as the fingers 32 make contact with the distal end 34 of the hip stem 14, the features within the cavity 26 (i.e., the push rod and/or the balloon) may cause the fingers 32 to radially deflect open and capture the distal end 34 of the hip stem 14 within the cavity 26.

In an alternative embodiment (not illustrated), the intramedullary device 10 may extend from a closed box of an otherwise conventional knee femoral component. The intramedullary device 10 may be formed with the knee femoral component or suitably attached to the knee femoral component. For example, the fastening means may engage the threaded portion 38 and/or the intramedullary device 10 to attach the intramedullary device 10 to the knee femoral component. Once attached, the proximal end 18 of the intramedullary device 10 may be inserted into the intramedullary canal of the femur. As the intramedullary device 10 is inserted into the intramedullary canal and the fingers 32 make contact with the distal end 34 of the hip stem 14, pressure applied by the hip stem 14 against the fingers 32 may again cause the fingers 32 to outwardly deflect in a radial direction and capture the distal end 34 of the hip stem 14 within the cavity 26. Alternatively, as the fingers 32 make contact with the distal end 34 of the hip stem 14, the features within the cavity 26 (i.e., the push rod and/or the balloon) may cause the fingers 32 to radially deflect open and capture the distal end 34 of the hip stem 14 within the cavity 26.

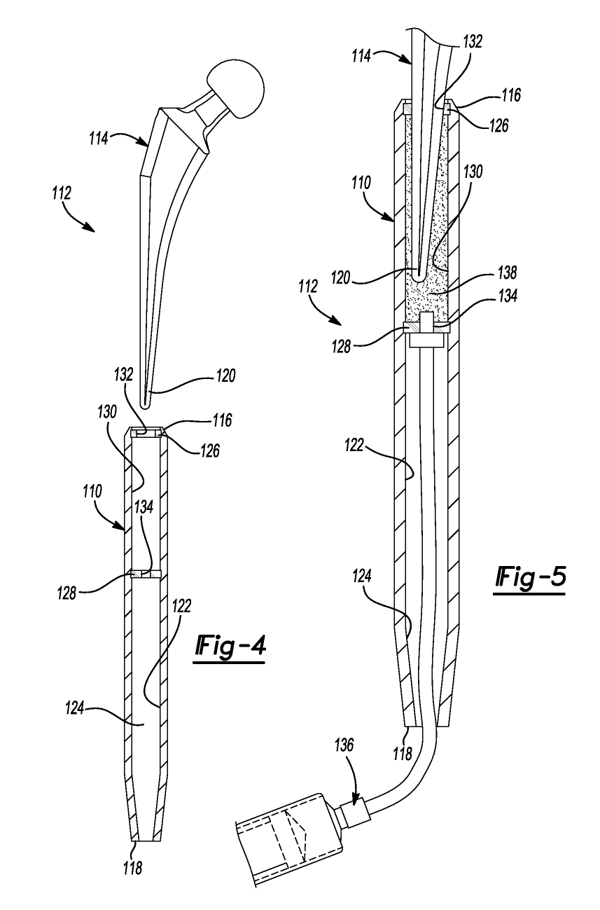

With reference to FIGS. 4 and 5, another intramedullary device for treating periprosthetic fractures constructed in accordance with the teachings of the present disclosure is illustrated and generally identified at reference numeral 110, which can be incorporated into an exemplary construct 112. In this particular exemplary construct 112, the intramedullary device 110 is shown associated with a conventional hip stem 114; however, it will be understood that the intramedullary device 110 may be used in connection with other implants and that the present teachings have application beyond the exemplary construct 112 illustrated in the drawings and described herein.

Similar to the intramedullary device 10 described above, the intramedullary device 110 is an elongated member fabricated from a material having requisite strength properties and suitable for implantation into the intramedullary canal of a patient's bone, including, but not limited to the femur, tibia and the humerus (none illustrated), for example. A proximal end 116 of the intramedullary device 110 can be configured to engage a distal end 120 of the hip stem 114. A distal end 118 of the intramedullary device 110 can be configured to pass through and/or engage an orthopedic implant, such as a knee femoral component, tibial tray component or a shoulder stem (none illustrated), for example.

A center bore 122 may extend longitudinally through the intramedullary device 110, between the proximal and distal ends 116, 118, thereby defining a cavity 124. At least one through bore (not illustrated) may extend through the intramedullary device 110 in a direction generally transverse to the center bore 122. The through bore(s) can provide for a fixation device(s), such as a plate, rod, screw, nail, etc., for example, to pass through the intramedullary device 110 and provide additional femoral fixation.

Within the cavity 124, the intramedullary device 110 may include a first barrier 126 and a second barrier 128 cooperating to form a chamber 130 therebetween. The barriers 126, 128 may be fabricated from a material similar to the material of the intramedullary device 110, or may be fabricated from a material having flexible sealing properties, such as a plastic or elastomeric material, for example. The first barrier 126 may include a first opening 132, and the second barrier 128 may include a second opening 134.

Referring specifically to FIG. 5, the distal end 120 of the hip stem 114 can be received in the chamber 130 of the intramedullary device 110 through the first opening 132 in the first barrier 126. An ancillary tool 136, such as an injection instrument or cement gun, for example, or, more specifically, a delivery nozzle or tip of the ancillary tool 136, can be inserted into the distal end 118 of the intramedullary device 110 and through the second opening 134 in the second barrier 128. As will be described in further detail below, with the distal end 120 of the hip stem 114 received in the chamber 130, the ancillary tool 136 can be configured to deliver or inject a cement or bonding mixture 138 into the chamber 130 to engage and secure the distal end 120 of the hip stem 114 within the chamber 130.

With continued reference to FIG. 5, a method for treating a periprosthetic fracture in the femur of a patient having an existing hip stem 114 will now be described. Using known techniques, the proximal end 116 of the intramedullary device 110 can be inserted in a retrograde direction into the intramedullary canal of the patient's femur until a portion of the distal end 120 of the hip stem 114 is received through the first opening 132 in the first barrier 126 and in the chamber 130. The ancillary tool 136 can be inserted into the distal end 120 of the intramedullary device 110 and through the second opening 134 in the second barrier 128. With the chamber 130 at least partially sealed at the upper and lower ends by the distal end 120 of the hip stem 114 and the ancillary tool 136 respectively, the bonding mixture 138 can be delivered or injected into the chamber 130 and surround the distal end 120 of the hip stem 114. As the bonding mixture 138 is curing, or once the bonding mixture 138 has cured, the ancillary tool 136 can be removed from the distal end 120 of the intramedullary device 110. Accordingly, the distal end 120 of the hip stem 114 is engaged and secured within the intramedullary device 110.

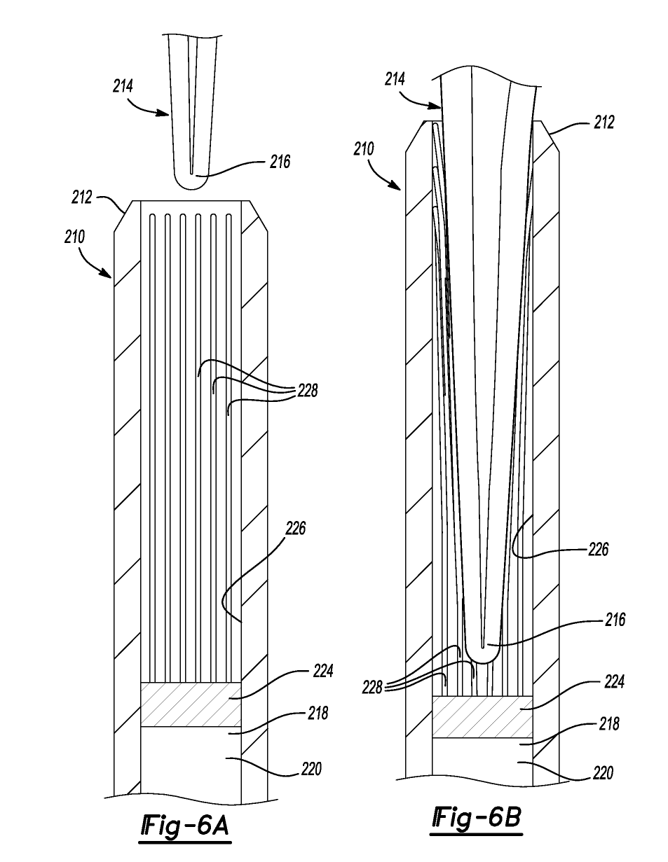

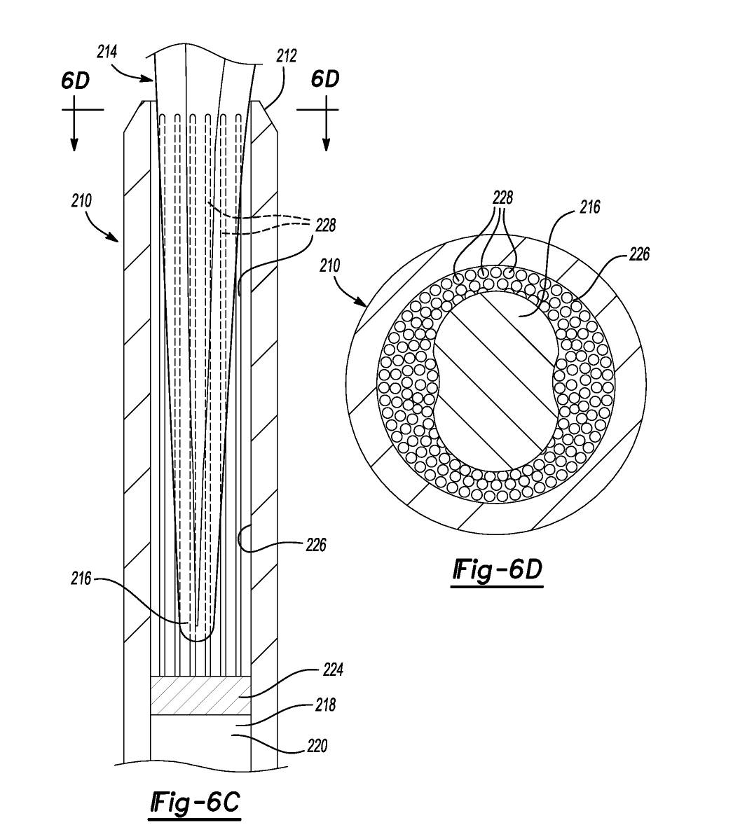

With reference to FIGS. 6A-6D, a proximal end 212 of another intramedullary device 210 for treating periprosthetic fractures constructed in accordance with the teachings of the present disclosure is illustrated. The intramedullary device 210 is shown associated with a distal end 216 of conventional hip stem 214; however, it will again be understood that the present teachings may be used in connection with other implants

Similar to the intramedullary devices 10, 110 described above, intramedullary device 210 is an elongated member fabricated from a material having requisite strength properties and suitable for implantation into the intramedullary canal of a patient's bone, including, but not limited to the femur, tibia and the humerus (none illustrated), for example. The proximal end 212 of the intramedullary device 210 can be configured to engage the distal end 216 of the hip stem 214. The distal end (not illustrated) of the intramedullary device 210 can be configured to pass through and/or engage an orthopedic implant, such as a knee femoral component, a tibial tray component or a shoulder stem (none illustrated).

A center bore 218 may extend longitudinally through the intramedullary device 210, thereby defining a cavity 220 within the intramedullary device 210. Within the cavity 220, a barrier or wall 224 may extend transversely to the center bore 218, thereby defining an open-ended chamber 226 at the proximal end 212 of the intramedullary device 210. With specific reference to FIG. 6A, a plurality of fingers or rods 228 in a first configuration may extend upwardly from the wall 224 toward the proximal end 212 of the intramedullary device 210. As shown in this first configuration, the rods 228 may extend generally straight and parallel to one another. The rods 228 may be fabricated from a shape-memory alloy, which may be referred to in the art as “memory metal.” A characteristic of a shape-memory alloy is that, generally, it “remembers” its original shape once it has been deformed and then heated. Stated another way, when a shape-memory alloy is deformed, and subsequently heated past a predetermined transfer temperature, the deformed shape-memory alloy returns to its original pre-deformed shape.

Referring specifically to FIG. 6B, the distal end 216 of the hip stem 214 can be configured to be received in the proximal end 212 of the intramedullary device 210 and positioned within the chamber 226 amongst the rods 228. To make room for the distal end 216 of the hip stem 214 within the chamber 226, the rods 228 may deform, bend, move, and/or flex from the first configuration shown in FIG. 6A to a second configuration shown in FIG. 6B. It is understood that when the rods 228 are in the second configuration shown in FIG. 6B, the distal end 216 of the hip stem 214 may still be moveable within the chamber 226 amongst the rods 228. However, as will be described further below, when the rods 228 are heated and try to return to their original de-formed configuration (i.e., generally straight and parallel to one another), the rods 228 may apply a pressure or force onto the distal end 216 of the hip stem 214, thereby engaging and securing the hip stem 214 within the chamber 226.

With continued reference to FIG. 6A-6D, a method for treating a periprosthetic fracture in the femur of a patient having an existing hip stem 214 will now be described. Using known techniques, the proximal end 212 of the intramedullary device 210 can be inserted in a retrograde direction into the intramedullary canal of the patient's femur until a portion of the distal end 216 of the hip stem 214 is received in the chamber 226 (FIG. 6B). Accordingly, as the distal end 216 of the hip stem 214 is received in the chamber 226, the rods 228 may deform, bend, move, and/or flex. Once the distal end 216 of the hip stem 214 is in an appropriate position within the chamber 226, sufficient heat from a conventional heat source (not illustrated) that is above the transfer temperature of the rods 228 can be applied to the intramedullary device 210 to try to return the rods 228 to their original pre-deformed shape. Alternatively, the transfer temperature of the rods 228 may be close to body temperature. Accordingly, with the intramedullary device 210 in the intramedullary canal of the patient's femur, body heat from the patient may function to try to return the rods 228 to their original pre-deformed shape. As the rods 228 try to return to their original de-formed configuration (i.e., generally straight and parallel to one another), the rods 228 may apply a pressure or force onto the hip stem 214, thereby engaging and capturing the hip stem 214 therein.

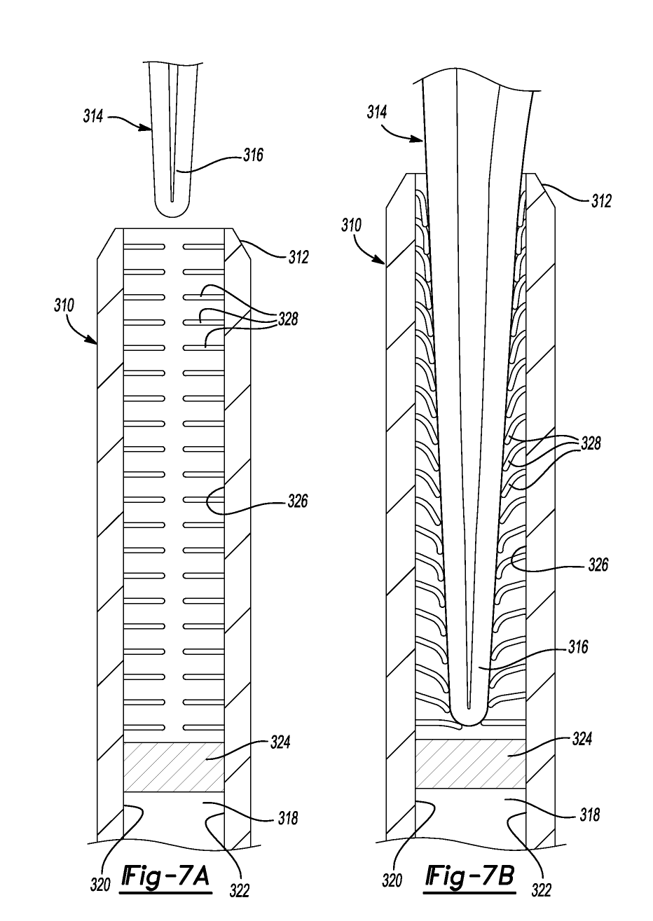

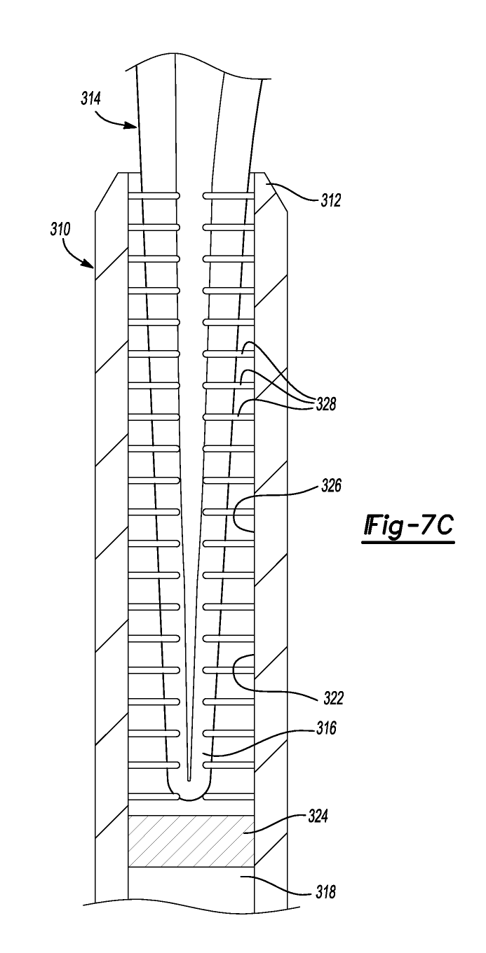

With reference to FIGS. 7A-7C, a proximal end 312 of another intramedullary device 310 for treating periprosthetic fractures constructed in accordance with the teachings of the present disclosure is illustrated. The intramedullary device 310 is shown associated with a distal end 316 of a conventional hip stem 314; however, it will again be understood that the present teachings may be used in connection with other implants.

Similar to the intramedullary devices 10, 110, 210 described above, intramedullary device 310 is an elongated member fabricated from a material having requisite strength properties and suitable for implantation into the intramedullary canal of a patient's bone, including, but not limited to the femur, tibia and the humerus (none illustrated), for example. The proximal end 312 of the intramedullary device 310 can be configured to engage the distal end 316 of the hip stem 314. The distal end (not illustrated) of the intramedullary device 310 can be configured to pass through and/or engage an orthopedic implant, such as a knee femoral component, a tibial tray component or a shoulder stem (none illustrated).

A center bore 318 may extend longitudinally through the intramedullary device 310, thereby defining a cavity 320 having an inner surface 322 within the intramedullary device 310. Within the cavity 320, a barrier or wall 324 may extend transversely to the center bore 318, thereby defining an open-ended chamber 326 at the proximal end 312 of the intramedullary device 310. With specific reference to FIG. 7A, a plurality of fingers or rods 328 in a first configuration may extend horizontally from the inner surface 322 of the chamber 326. In this first configuration, the rods 328 may extend generally straight and parallel to one another and generally perpendicular to the center bore 318. Similar to the rods 228 described above, the rods 328 may be fabricated from a shape-memory alloy, which may also be known in the art as “memory metal.” As described above, when a shape-memory alloy is deformed, and then heated past a predetermined transfer temperature, the deformed shape-memory alloy tries to its original pre-deformed shape.

Referring specifically to FIG. 7B, the distal end 316 of the hip stem 314 can be configured to be received in the proximal end 312 of the intramedullary device 310 and positioned within the chamber 326 amongst the rods 328. To make room for the distal end 316 of the hip stem 314 within the chamber 326, the rods 328 may deform, bend, move, and/or flex from the first configuration shown in FIG. 7A to a second configuration shown in FIG. 7B. It is understood that when the rods 328 are in the second configuration, the distal end 316 of the hip stem 314 may be moveable within the chamber 326 amongst the rods 328. However, as will be described further below, when the rods 328 are heated and try to return to their original deformed configuration (i.e., generally straight and parallel to one another), the rods 328 may apply a pressure or force onto the with the distal end 316 of the hip stem 314 in the chamber 326, thereby engaging the hip stem 314 therein.

With continued reference to FIG. 7A-7C, a method for treating a periprosthetic fracture in the femur of a patient having an existing hip stem 314 will now be described. Using known techniques, the proximal end 312 of the intramedullary device 310 can be inserted in a retrograde direction into the intramedullary canal of the patient's femur until a portion of the distal end 316 of the hip stem 314 is received in the chamber 326 (FIG. 7B). Accordingly, as the distal end 316 of the hip stem 314 is received into the chamber 326, the rods 328 may deform, bend, move, and/or flex. Once the distal end 316 of the hip stem 214 is in an appropriate position within the chamber 326, sufficient heat form a heat source (not illustrated) that is above the transfer temperature of the rods 328 can be applied to the intramedullary device 310 to try to return the rods 328 to their original pre-deformed shape. Alternatively, the transfer temperature of the rods 328 may be close to the body temperature of the patient. With the intramedullary device 310 in the intramedullary canal of the patient's femur, the patient's body heat may function to try to return the rods 328 to their original pre-deformed shape. Accordingly, as the rods 328 try to return to their original de-formed configuration (i.e., generally straight and parallel to one another), the rods 328 may apply a pressure or force onto the distal end 316 of the hip stem 314, thereby engaging the hip stem 314 therein.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. An intramedullary device comprising:
a tubular body extending along a center longitudinal axis, the tubular body including a first end and a second end, the first end configured to engage a stem of an implant.

2. The intramedullary device of claim 1, wherein the first end includes fingers, the fingers configured to radially deflect from a first configuration to a second configuration, wherein the fingers engage the stem of the implant in the second configuration.

3. The intramedullary device of claim 2, wherein the tubular body further includes a cavity at the first end thereof, the fingers extending from a surface disposed within the cavity.

4. The intramedullary device of claim 3, wherein in the fingers are fabricated from a shape memory alloy having a transition temperature.

5. The intramedullary device of claim 4, wherein in the first configuration each of the fingers extend along a longitudinal axis that is generally parallel to the center longitudinal axis of the tubular body.

6. The intramedullary device of claim 4, wherein in the first configuration each of the fingers extend along a longitudinal axis that is generally perpendicular to the longitudinal axis of the tubular body.

7. The intramedullary device of claim 4, wherein the fingers deflect from the first configuration to the second configuration once the fingers are exposed to a temperature at or above the transition temperature.

8. The intramedullary device of claim 1, wherein the tubular body further includes a cavity at the first end, the cavity includes a first barrier at an upper end thereof, the stem of the implant is configured to be received through an opening in the first barrier and engaged in the cavity.

9. The intramedullary device of claim 8, wherein a bonding mixture is disposed within the cavity between the first barrier and a second barrier located at a lower end of the cavity, the bonding mixture configured to engage the stem of the implant.

10. The intramedullary device of claim 9, wherein the second barrier defines an opening for receiving a tool to deliver the bonding mixture to the cavity.

11. An implantable construct comprising;
a knee implant;
a hip implant including an intramedullary hip stem; and
an intramedullary tube extending along a longitudinal axis from a proximal end to a distal end, the proximal end of the intramedullary tube including a cavity configured to receive and engage a distal end of the intramedullary hip stem therein, the distal end of the intramedullary tube configured to engage the knee implant.

12. The implantable construct of claim 11, wherein the intramedullary tube includes a cementing compound within the cavity, the cementing compound configured to harden and engage the distal end of the intramedullary hip stem received in the cavity.

13. The implantable construct of claim 12, wherein the cavity includes a first barrier proximate an upper end thereof and a second barrier proximate a lower end thereof, the distal end of the intramedullary hip stem is configured to be received through the first barrier, the second barrier configured to receive a tool for supplying the cementing compound into the cavity.

14. The implantable construct of claim 11, wherein fingers extending from a wall within the cavity of the intramedullary tube are configured to deflect and engage the distal end of the intramedullary hip stem.

15. The implantable construct of claim 14, wherein the fingers are fabricated from a shape memory alloy having a transition temperature, in a first configuration the fingers do not engage the distal end of the intramedullary hip stem when the distal end of the intramedullary hip stem is received in the cavity, in a second configuration once the fingers are exposed to a temperature near the known transition temperature value the fingers deflect and engage the distal end of the intramedullary hip stem.

16. The intramedullary device of claim 15, wherein in the first configuration each of the fingers extend along a longitudinal axis that is generally parallel to the longitudinal axis of the intramedullary tube.

17. The intramedullary device of claim 15, wherein in the first configuration each of the fingers extend along a longitudinal axis that is generally perpendicular to the longitudinal axis of the intramedullary tube.

18. A method of treating a bone, the method comprising:
providing a tubular member;
inserting the tubular member into an intramedullary canal of a patient; and
engaging a first end of the tubular member with a stem of an implant while the stem is in the intramedullary canal of the patient.

19. The method of claim 18, wherein inserting the tubular member into the intramedullary canal of the patent includes passing the tubular member through an opening in a knee component.

20. The method of claim 18, wherein engaging the first end of the tubular member with the stem of the implant further includes inserting the stem of the implant into a cavity located at the first end of the tubular member.

21. The method of claim 20, wherein engaging the first end of the tubular member with the stem of the implant includes engaging the stem of the implant with fingers provided on the tubular member.

22. The method of claim 20, wherein the fingers extend from a wall within the cavity, the fingers being configured to deflect when the stem of the implant is inserted into the cavity to engage the stem of the implant.

23. The method of claim 22, further comprising exposing the fingers to a temperature above a predetermined transition temperature once the stem of the implant is inserted into the cavity to deflect the fingers and engage the stem of the implant.

24. The method of claim 20, wherein engaging the first end of the tubular member with the stem of the implant further includes surrounding the stem of the implant with a cement disposed within the cavity.

25. The method of claim 24, further comprising:
inserting a tool into a second end of the tubular member and injecting the cement into the cavity, the cement surrounding the stem of the implant inserted into the cavity; and
removing the tool from the second end of the tubular member.

26. The method of claim 25, further comprising:
inserting the stem of the implant into the cavity through a first barrier located proximate an upper end of the cavity;
inserting the tool into the cavity through a second barrier located proximate a lower end of the cavity to inject the cement into the cavity.
